(19) United States
(12) Patent Application Publication
(10) Pub. No.: US 2026/0284328 A1
(43) Pub. Date: Sep. 24, 2026

(54) NASAL DELIVERY DEVICE FOR DELIVERING A MEDICATION INTO THE NOSE, AND METHODS OF TREATING A MEDICAL CONDITION USING THE NASAL DELIVERY DEVICE

(71) Applicant: Raymond J. Oenbrink, DO, Holly Hill, FL (US)

(72) Inventor: Raymond J. Oenbrink, DO, Holly Hill, FL (US)

(21) Appl. No.: 19/332,798

(22) Filed: Sep. 18, 2025

Related U.S. Application Data
(60) Provisional application No. 63/775,412, filed on Mar. 21, 2025.

(51) Int. Cl. A61M 11/00

(52) U.S. Cl. CPC A61M 11/006; A61M 2202/0468; A61M 2205/02

(57) ABSTRACT

Delivery devices, and methods of using the delivery devices, for use in treating or alleviating a medical condition. Embodiments of the invention provide devices for delivering a medication, such as lidocaine, to a user's nose, for the treatment or alleviation of one or more symptoms of a migraine.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/775,412, entitled, “NASAL DELIVERY DEVICE FOR DELIVERING A MEDICATION INTO THE NOSE, AND METHODS OF TREATING A MEDICAL CONDITION USING THE NASAL DELIVERY DEVICE”, filed on Mar. 21, 2025. The contents of the above referenced application are incorporated herein by reference in its entirety.

### FIELD OF THE INVENTION

Embodiments of the disclosure relate to delivery devices, and methods of using the delivery devices; to devices and methods of treating medical conditions or disorders; and more particularly, to delivery devices for delivery of a medication to the nose for treating migraines, and methods related thereto.

### BACKGROUND OF THE INVENTION

A migraine is a common neurologic disorder characterized by a severe, often disabling headache. Often times, an individual with a migraine suffers from intense, throbbing pain, blurred or other vision changes, sensitivity to light and sound, nausea, dizziness, and fatigue. With such disabling pain, migraines negatively affect a person's quality of life, the ability to work productively, or enjoy various leisure activities. Typical options for treating a migraine include oral administration of various drugs, such as nonsteroidal anti-inflammatory drugs (NSAID), antiemetics, triptans, ditans, gepants, and dihydroergotamine. These medications are not always effective. In addition, many of these drugs can cause severe side effects. What is needed, therefore, is additional migraine intervention that provides faster, ideally within minutes, relief.

### SUMMARY OF THE INVENTION

Embodiments disclosed herein concern delivery devices, and methods of using the delivery devices, for use in treating or alleviating a medical condition. Specific embodiments of the invention provide devices for delivering a medication, such as lidocaine, into a user's nose, for the treatment or alleviation of one or more symptoms of a medical condition, such as migraine.

Accordingly, it is an objective of the invention to teach devices for delivering medication.

It is a further objective of the invention to provide methods of delivering medication.

It is yet another objective of the invention to provide devices for delivering medication to a user's sphenopalatine ganglion (SPG) via the inferior floor of the nares/nostrils.

It is a still further objective of the invention to provide methods for delivering medication to sphenopalatine ganglion (SPG) via the inferior floor of the nares/nostrils.

It is a further objective of the invention to provide devices for delivering medication to a user's sphenopalatine ganglion (SPG) via the inferior floor of the nares/nostrils for treating or alleviating migraine.

It is yet another objective of the invention to provide methods for delivering medication to a user's sphenopalatine ganglion (SPG) via the inferior floor of the nares/nostrils for treating or alleviating migraine.

It is a still further objective of the invention to provide devices for delivering medication to each individual nostril of a user's nose for treating or alleviating migraine headaches.

It is a further objective of the invention to provide methods for delivering medication to each individual nostril of a user's nose for treating or alleviating migraine headaches.

It is yet another objective of the invention to provide devices for delivering medication to each individual nostril of a user's nose for treating or alleviating migraine headaches, wherein back flow of the medication is prevented or minimized.

It is a still further objective of the invention to provide methods for delivering medication to each individual nostril of a user's nose for treating or alleviating migraine headaches, wherein back flow of the medication is prevented or minimized.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of an illustrative embodiment of a device for nasal delivering of a medication for the treatment or alleviation of one or more symptoms of a medical condition, such as lidocaine;

FIG. 2A illustrates the device for nasal delivering of a medication for the treatment or alleviation of one or more symptoms of a medical condition illustrated in FIG. 1, shown without plungers;

FIG. 2B is a bottom view of the device for nasal delivering of a medication for the treatment or alleviation of one or more symptoms of a medical condition illustrated in FIG. 2;

FIG. 3 illustrates insertion of an embodiment of a nasal passage sealing member into a nostril;

FIG. 4 illustrates an embodiment of the nasal passage sealing member inserted into the left and right nostrils;

FIG. 5 is an embodiment of the device for delivering medication illustrated in FIG. 1, using a single plunger;

FIG. 6 illustrates an embodiment of the device for delivering medication for the treatment or alleviation of one or more symptoms of a medical condition, illustrating an alternative fluid administering unit;

FIG. 7 illustrates the embodiment of the device for delivering medication for the treatment or alleviation of one or more symptoms of a medical condition;

FIG. 8 illustrates an embodiment of the device for delivering medication for the treatment or alleviation of one or more symptoms of a medical condition, illustrating an alternative fluid administering unit;

FIG. 9 illustrates an embodiment of the device for delivering medication for the treatment or alleviation of one or more symptoms of a medical condition, illustrating an alternative fluid administering unit;

FIG. 10 illustrates an embodiment of the device for delivering medication for the treatment or alleviation of one or more symptoms of a medical condition, illustrating an alternative fluid administering unit; and

FIG. 11 illustrates an embodiment of the device for delivering medication for the treatment or alleviation of one or more symptoms of a medical condition, illustrating an alternative fluid administering unit.

### DETAILED DESCRIPTION OF THE INVENTION

While the present invention is susceptible of embodiment in various forms, there is shown in the drawings and will hereinafter be described non-limiting embodiments with the understanding that the present disclosure is to be considered an exemplification of the present invention and is not intended to limit the invention to the specific embodiments illustrated.

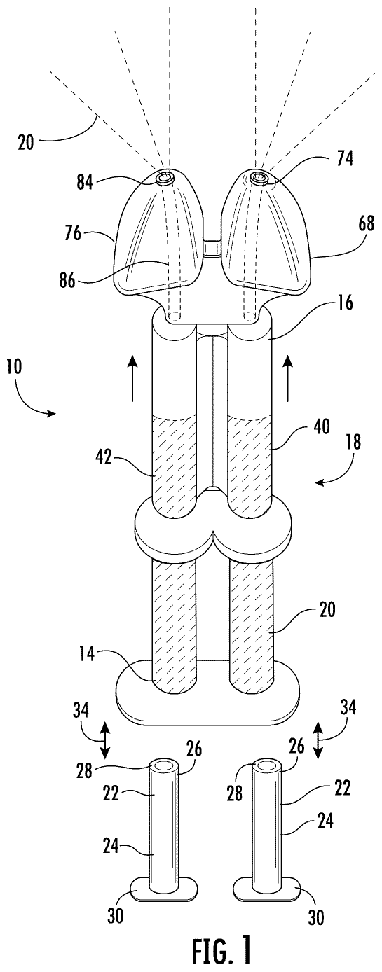

Referring to FIGS. 1-12, embodiments of a device for delivering a medication, such as lidocaine, for the treatment or alleviation of one or more symptoms of a medical condition, such as a migraine, are provided. Referring specifically to FIGS. 1-5, an embodiment of the device for delivering a medication for the treatment or alleviation of one or more symptoms of a medical condition, referred to generally as a nasal delivery device 10, is shown. The nasal delivery device 10 is constructed and arranged to deliver a fluid, such as a viscous fluid, including one or more fluids or viscous fluids, (or combinations of fluids or viscous fluids), such as a medication, illustrated and described herein as lidocaine, to a user's sphenopalatine ganglion (SPG) via the inferior floor of the nares/nostrils to provide relief, i.e. treat or alleviate one or more symptoms of a medical condition, illustrated and described herein as a migraine. As such, the nasal delivery device 10 provides targeted delivery of lidocaine to an individual's sphenopalatine ganglion (SPG) via the inferior floor of the nares/nostrils, simultaneously to each individual nostril, or to one specific nostril, with minimal or no leakage or back flow (fluid injected into the nostrils but flowing back out due to internal nose pressure). Such action ensures that the lidocaine is delivered in the correct amounts, to the correct place (i.e., to numb or desensitize the cells of the sphenopalatine ganglion (SPG)), and for the correct amount of time, thus ensuring maximum effectiveness. The nasal delivery device 10 therefore allows injected lidocaine to pool on the nostril floor above the SPG.

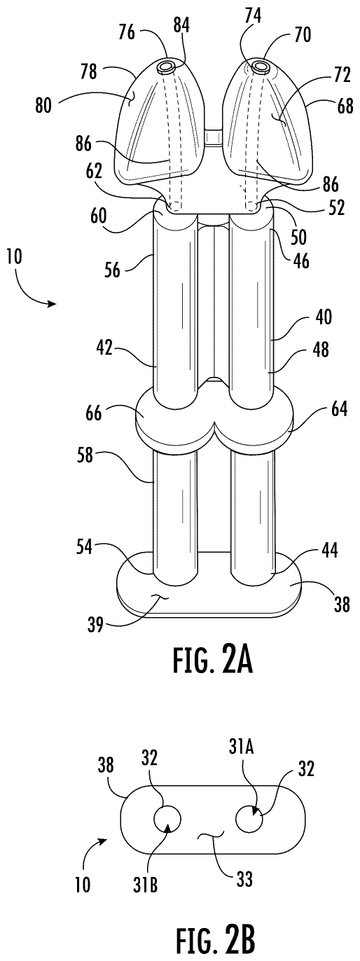

The nasal delivery device 10 comprises a double barrel delivery mechanism, having a first end 14, a second end 16, and a main body 18 separating the first end 14 and the second end 16. The nasal delivery device 10 is designed to dispense (and in some embodiments store) the lidocaine 20 into to the user's nose. Lidocaine 20 may be in any injectable form such as a liquid, gel, or other form known to one of skill in the art. The nasal delivery device 10 includes a base member 38 (see FIG. 2A) designed to receive plungers 22 and/or support a user's fingers when in use. Each of the plungers 22 (creates a force to expel any liquid agent) has an elongated body 24 having a tip 26. The tip 26 may include a pliable/flexible stopper 28. Opposite the tip 26, the plunger 22 may include a finger flange 30 for supporting a user's finger. The plungers 22 are designed to be inserted into the interior sections 31A or 31B (see FIG. 2B) via openings 32 on the bottom surface 33 of the base member 38, moving linearly (back and forth, see double arrows 34) based on a directional force (pushing or pulling of the plunger(s) 22) being applied. By using two plungers 22, the user can insert the lidocaine 20 in both nostrils of the user, or just one nostril (if, for example, the migraine headache pain is located on one side).

Extending upwardly from the outer surface 39 of the base member 38 is a first fluid delivery member 40 and a second fluid delivery member 42. The first fluid delivery member 40 and the second fluid delivery member 42 are supported by the base 38. The first fluid delivery member 40 comprises a first end 44, a second end 46, and a body 48 separating said first end 44 and said second end 46. The body 48 is illustrated assuming an elongated, tube or cylindrical-like shape, but could assume any shape or size necessary to function. The body 48 extends away from the base member 38 and terminates in a tip 50. The tip 50 is shown having a rounded or ball-like shape, with an opening 52. Such shape is illustrative, as the tip 50 could assume other shapes. The main body 48 includes a hollow or partially hollow interior 31A, thus allowing the lidocaine 20 to move within and be delivered out through the opening 52. In certain embodiments, lidocaine 20 can be stored within.

The second fluid delivery member 42 comprises a first end 54, a second end 56, and a body 58 separating said first end 54 and said second end 56. The body 58 is illustrated assuming an elongated, tube or cylindrical-like shape, but could assume any shape or size necessary to function. The body 58 extends away from the base member 38 and terminates in a tip 60. The tip 60 is shown having a rounded or ball-like shape, with an opening 62. Such shape is illustrative, as the tip 60 could assume other shapes. The body 58 includes a hollow or partially hollow interior 31B, thus allowing the lidocaine to move within and be delivered out through the opening 62. In certain embodiments, lidocaine 20 can be stored within. A pair of knob-like structures, 64 and 66, extend around a perimeter of the first fluid delivery member 40 and second fluid delivery member 42, connecting in the area between the two structures. The knob-like structures, 64 and 66 provide a gripping or stabling structure for the user's fingers to rest thereon when in use dispensing the lidocaine 20. The knob-like structures, 64 and 66 may also provide support to prevent the first fluid delivery member 40 or the second fluid delivery member 42 from moving.

Fluidly connected to the first fluid delivery member 40 is a first nasal passage sealing member 68 (may also be referred to as nasal adaptor plug 68). The first nasal passage sealing member 68 is constructed and arranged to provide a mechanism to maintain the amount of injected lidocaine in the nostril. The first nasal passage sealing member 68 may assume a convex, conical, or hemispheric shape.

The first nasal passage sealing member 68 may be made of a pliable or flexible material. The nasal passage sealing member 68 comprises a body 70 having an outer surface 72 and a bottom surface 73 (see FIG. 4). The body 70 has an interior fluid passageway, illustrated herein as a fluid line 86. The fluid line 86 spans the length of the interior of the body 70, terminating in opening, illustrated herein as a dispensing nipple or nozzle 74. The fluid line 86, as well as the dispensing nipple or nozzle 74, is configured to allow passage of the lidocaine. The dispensing nipple or nozzle 74 allows for the lidocaine to be injected out of the first nasal passage sealing member 68. The fluid dispensing nipple or nozzle 74 may be made of a pliable material. The first nasal passage sealing member 68 body may have a size and shape to mirror the size and shape of the opening of an individual's nostril. When placed within the opening of the individual's nostril, the first nasal passage sealing member 68 prevents any lidocaine from flowing out of the nostril it was inserted therein.

Fluidly connected to the second fluid delivery member 42 is a second nasal passage sealing member 76 (may also be referred to as nasal adaptor plug 76). The second nasal passage sealing member 76 is constructed and arranged to provide a mechanism to maintain the amount of injected lidocaine in the nostril. The second nasal passage sealing member 76 may assume a convex, conical, or hemispheric shape.

The second nasal passage sealing member 76 may be made of a pliable or flexible material. The second nasal passage sealing member 76 comprises a body 78 having a first surface 80 and a bottom surface 82 (see FIG. 4). The body 78 has an interior fluid line 86. The fluid line 86 spans the length of the interior of the body 78, terminating in opening, illustrated herein as a second dispensing nipple or nozzle 74. The fluid line 86, as well as the second dispensing nipple or nozzle 84, is configured to allow passage of the lidocaine. The second fluid dispensing nipple or nozzle 84 allows for the lidocaine to be injected out of the second nasal passage sealing member 76. The second fluid dispensing nipple or nozzle 84 may be made of a pliable material. The second nasal passage sealing member body 76 may be sized and shaped to mirror the size and shape of the opening of an individual's nostril. When placed within the opening of the individual's nostril, the second nasal passage sealing member 76 prevents the lidocaine from flowing out of the nostril it was inserted therein. Both the first nasal passage sealing member 68 and the second nasal passage sealing member 76 fluidly connect to the first fluid delivery member 40 and the second fluid delivery member 42 via the fluid line 86.

The first nasal passage sealing member 68 and the second nasal passage sealing member 76 may be made of a material that is malleable so that it may be reduced in size or shape upon an applied pressure (prior to insertion into the user's nostrils) and expanding upon insertion or placement into the into the user's nostrils, thus forming a pressure fit seal. The first nasal passage sealing member 68 and the second nasal passage sealing member 76 are shown as a single unit, operationally attaching to of being integrally formed with or to the first fluid delivery member 40 and the second fluid delivery member 42.

Fluid line 86 may be integrally formed as part of the first fluid delivery member 40 and the second fluid delivery member 42 or made as a separate unit and secured or attached thereto. In either case, they are designed to allow for fluid to flow or move within, transferring fluid from the inner sections of first fluid delivery member 40 and the second fluid delivery member 42, through the first nasal passage sealing member 68 and the second nasal passage sealing member 76, and out into the user's nose.

The fluid line 86 may be made of any medical grade material, such as medical grade silicone or thermoplastic elastomer (TPE). The fluid line 86 may be made of a rigid material, a semi-rigid material (tubing), or flexible material (such as a hose).

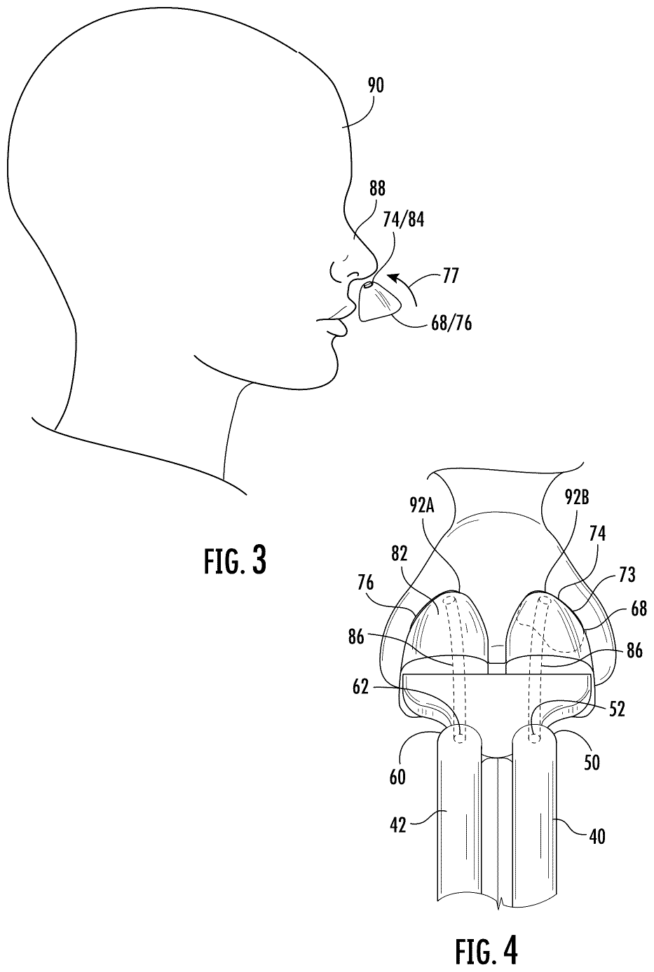

In use, the first nasal passage sealing member 68 and the second nasal passage sealing member 76 would be inserted into the nose 88 of an individual 90, see FIG. 3. The first nasal passage sealing member 68 and the second nasal passage sealing member 76 are positioned so that the first fluid dispensing nipple or nozzle 74 and the second fluid dispensing nipple or nozzle 84 are placed on or inside the nasal cavity (see arrows 77). Once the first nasal passage sealing member 68 and the second nasal passage sealing member 76 are inserted into and/or cover each nostril opening (see FIGS. 4), 92A (right nostril opening) and 92B left nostril opening), with the second, opposing surfaces 73 and 82 facing out, the user 90 applies a pressure, by pushing the plungers 22 inwardly. This action causes the lidocaine, such as lidocaine 2-5%, 3 cc/ml per nostril/nare, to flow out from the first fluid delivery member 40 and the second fluid delivery member 42, into the first nasal passage sealing member 68 and the second nasal passage sealing member 76 via fluid lines 86. The lidocaine 20 is dispensed into the nasal cavity via first fluid dispensing nipple or nozzle 74 and the second fluid dispensing nipple or nozzle 84.

In cases where the lidocaine is pre-loaded into the nasal delivery device 10, the nasal delivery device 10 could include a seal (such as a nonporous material or other material that does not allow fluids to transfer or pass through), placed over the first fluid dispensing nipple or nozzle 74 and the second fluid dispensing nipple or nozzle 84 to prevent the lidocaine stored within from exiting out prematurely. Applying a sufficient force to the cover would result in their removal.

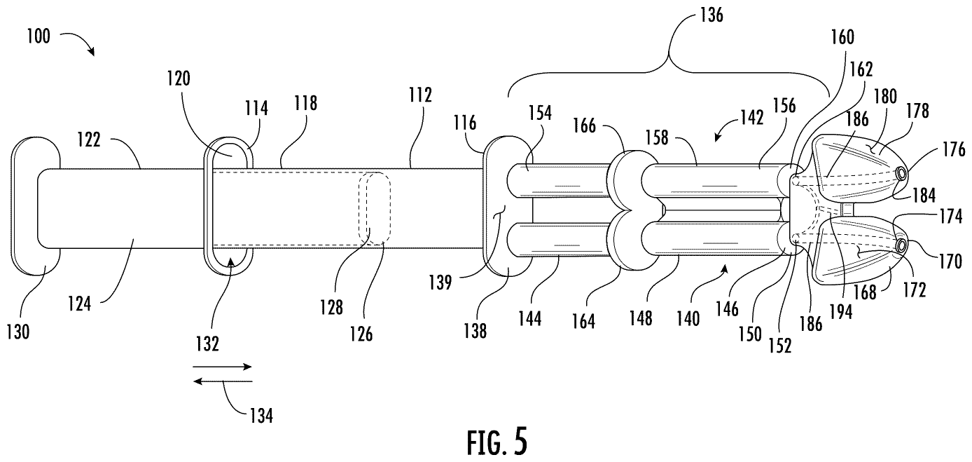

Referring to FIG. 5, an alternative embodiment of the device for nasal delivery of a medication for the treatment or alleviation of one or more symptoms of a medical condition, referred to generally as nasal delivery device 100, is shown. The nasal delivery device 100 functions in the same manner as that of nasal delivery device 10 and comprises many of the same structures. The nasal delivery device 100 differs structurally from the nasal delivery device 10 in that the nasal delivery device 100 uses a single barrel body and a single plunger to deliver the lidocaine. Alternatively, the nasal delivery device 100 differs may be designed as a double barrel body, using two plungers to store to deliver the lidocaine.

The nasal delivery device 100 comprises a barrel member 112 having a first end 114, a second end 116, and a body 118 separating the first end 114 and the second end 116. The body 118 assumes a shape, such as an elongated, generally cylindrical shape, and has an interior section 120 for storing the lidocaine therein. The interior section 120 is also sized and shaped to receive and store therein a single plunger 122. The plunger 122 has an elongated body 124 having a tip 126. The tip 126 may include a rubber stopper 128. Opposite the tip 126, the plunger 122 may include a finger flange 130 for supporting a user's finger. The plunger 122 is designed to be inserted into the interior section 120 via opening 132, moving linearly (back and forth, see double arrows 134) based on a directional force (pushing or pulling of the plunger 122) being applied.

Attached to or integrally formed as part of the second end 116 is a fluid administering unit 136. The fluid administering unit 136 comprises a base member 138. Extending upwardly from the outer surface 139 of the base member 138 is a first fluid delivery member 140 and a second fluid delivery member 142. The first fluid delivery member 140 and the second fluid delivery member 142 are supported by the base 138. The first fluid delivery member 140 comprises a first end 144, a second end 146, and a body 148 separating the first end 144 and the second end 146. The body 148 is illustrated assuming an elongated, tube or cylindrical-like shape, but could assume any shape or size necessary to function. The body 148 extends away from the base member 138 and terminates at a tip 150. The tip 150 is shown having a rounded or ball-like shape, with an opening 152. The main body 148 includes a hollow or partially hollow interior (same as interior 31A) thus allowing lidocaine to move within and be delivered out through the opening 152. In certain embodiments, lidocaine can be stored within.

The second fluid delivery member 142 comprises a first end 154, a second end 156, and a body 158 separating the first end 154 and the second end 156. The body 158 is illustrated assuming an elongated, tube or cylindrical-like shape, but could assume any shape or size necessary to function. The body 158 extends away from the base member 138 and terminates in a tip 160. The tip 160 is shown having a rounded or ball-like shape, with an opening 162. The body 158 includes a hollow or partially hollow interior (similar to that described for 31B) thus allowing the lidocaine to move within and be delivered out through the opening 162. In certain embodiments, lidocaine can be stored within. A pair of knob-like structures, 164 and 166, extend around a perimeter of the first fluid delivery member 140 and second fluid delivery member 142, connecting in the area between the two structures. The knob-like structures, 164 and 166 provide a gripping or stabling structure for the user's fingers to rest thereon when in use dispensing the lidocaine. The knob-like structures, 164 and 166, may also provide support to prevent the first fluid delivery member 140 or the second fluid delivery member 142 from moving.

The first nasal passage sealing member 168 may be made of a pliable or flexible material. The nasal passage sealing member 168 comprises a body 170 having an outer surface 172 and a bottom surface (not illustrated, but same as 73 illustrated in FIG. 4). The body 170 has an interior fluid passageway, illustrated herein as a fluid line 186. The fluid line 86 spans the length of the interior of the body 170, terminating in opening, illustrated herein as a dispensing nipple or nozzle 174. The fluid line 186, as well as the dispensing nipple or nozzle 174, is configured to allow passage of the lidocaine. The dispensing nipple or nozzle 174 allows for the lidocaine to be injected out of the first nasal passage sealing member 168. The fluid dispensing nipple or nozzle 174 may be made of a pliable material. The first nasal passage sealing member 168 body may have a size and shape to mirror the size and shape of the opening of an individual's nostril. When placed within the opening of the individual's nostril, the first nasal passage sealing member 168 prevents any lidocaine from flowing out of the nostril it was inserted therein

Fluidly connected to the second fluid delivery member 142 is a second nasal passage sealing member 176 (may also be referred to as nasal adaptor plug 176). The second nasal passage sealing member 76 is constructed and arranged to provide a mechanism to maintain the amount of injected lidocaine in the nostril. The second nasal passage sealing member 176 may assume a convex, conical, or hemispheric shape.

The second nasal passage sealing member 176 may be made of a pliable or flexible material. The second nasal passage sealing member 176 comprises a body 178 having a first surface 80 and a bottom surface (not illustrated but similar to 82 shown in FIG. 4). The body 178 has an interior fluid line 186. The fluid line 186 spans the length of the interior of the body 178, terminating in opening, illustrated herein as a second dispensing nipple or nozzle 174. The fluid line 186, as well as the second dispensing nipple or nozzle 184, is configured to allow passage of the lidocaine. The second fluid dispensing nipple or nozzle 184 allows for the lidocaine to be injected out of the second nasal passage sealing member 176. The second fluid dispensing nipple or nozzle 184 may be made of a pliable material. The second nasal passage sealing member body 176 may be sized and shaped to mirror the size and shape of the opening of an individual's nostril. When placed within the opening of the individual's nostril, the second nasal passage sealing member 176 prevents the lidocaine from flowing out of the nostril it was inserted therein. Both the first nasal passage sealing member 168 and the second nasal passage sealing member 176 fluidly connect to the first fluid delivery member 140 and the second fluid delivery member 42 via the fluid line 186.

The nasal delivery device 100 functions in the same manner as that described for the nasal delivery device 10, deferring in the use of the single barrel member 112 as an initial source for holding the lidocaine. As such the user would apply force to the plunger 122, moving the plunger 122 into the body 118, causing the lidocaine to flow into the fluid administering unit 136. In use, the first nasal passage sealing member 168 and the second nasal passage sealing member 176 would be inserted into the nose of an individual in need of a spray of lidocaine. In an illustrative example, lidocaine 2-5%, 3 cc/ml per nostril/nare is administered. The first nasal passage sealing member 168 and the second nasal passage sealing member 176 are positioned so that the first fluid dispensing nipple or nozzle 174 and the second fluid dispensing nipple or nozzle 184 are placed on or inside the nasal cavity. Once the first nasal passage sealing member 168 and the second nasal passage sealing member 176 are inserted into and/or cover each nostril openings, the user applies a pressure by pushing the plunger 122 inwardly. This action causes the lidocaine into the fluid administering unit 136. The lidocaine moves into and out from the first fluid delivery member 140 and the second fluid delivery member 142, into the fluid lines 186 of the first nasal passage sealing member 168 and the second nasal passage sealing member 176. The lidocaine is dispensed into the nasal cavity via first fluid dispensing nipple or nozzle 174 and the second fluid dispensing nipple or nozzle 184. The inserted the first nasal passage sealing member 168 and the second nasal passage sealing member 176 prevent the lidocaine from flowing out of the nose.

In certain embodiments, the nasal delivery device 100 may include a seal (not shown) covering opening 152 and opening 162 to prevent the lidocaine stored within from exiting out prematurely. In cases where the lidocaine is pre-loaded into the nasal delivery device 100, the nasal delivery device 100 could include a tab 194 (see FIG. 5). The tab 194 may have ends which are secured to the seals. Applying a sufficient force to the tab 194 would result in the removal to the seals.

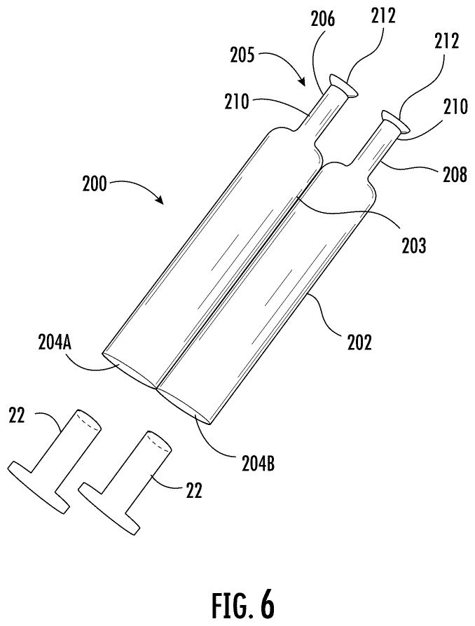

Referring to FIGS. 6-11, alternative embodiments of the device for delivering a medication for the treatment or alleviation of one or more symptoms of a medical condition are shown. Each of these devices functioning in the same general manner that described for nasal delivery device 10 or the nasal delivery device 100. The embodiments described differ mostly in using 1) either a single barrel, single plunger, or multiple/divided barrels, multiple plunger delivery systems, or 2) an alternative fluid administering unit. While each structure(s) is described for the specific embodiment being illustrated, any of the embodiments described can be adapted to include any individual structure(s) described and taught in any of the embodiments.

Referring to FIG. 6, the device for delivering a medication for the treatment or alleviation of one or more symptoms of a medical condition, referred to generally as nasal delivery device 200, is shown. The nasal delivery device 200 includes a body 202, illustrated as a double-barreled body, configured to receive two plungers 22. The double-barreled body 202 comprises a dividing wall 203, separating the double-barreled body 202 into two separate interior sections, 204A and 204B. A fluid administering unit 205 includes a first fluid delivery member 206 and a second fluid delivery member 208 positioned at the opposite end of the nasal delivery device 200 from where the plungers 22 are inserted. The first fluid delivery member 206 and the second fluid delivery member 208 each include a fluid passageway section 210 (allowing the fluid to move from the double-barreled body 202) and a nostril engagement member 212. The nostril engagement member, shown as a cup, is configured to be inserted into or rest just outside of the nostril of the user, directing the fluid inside of the nostril.

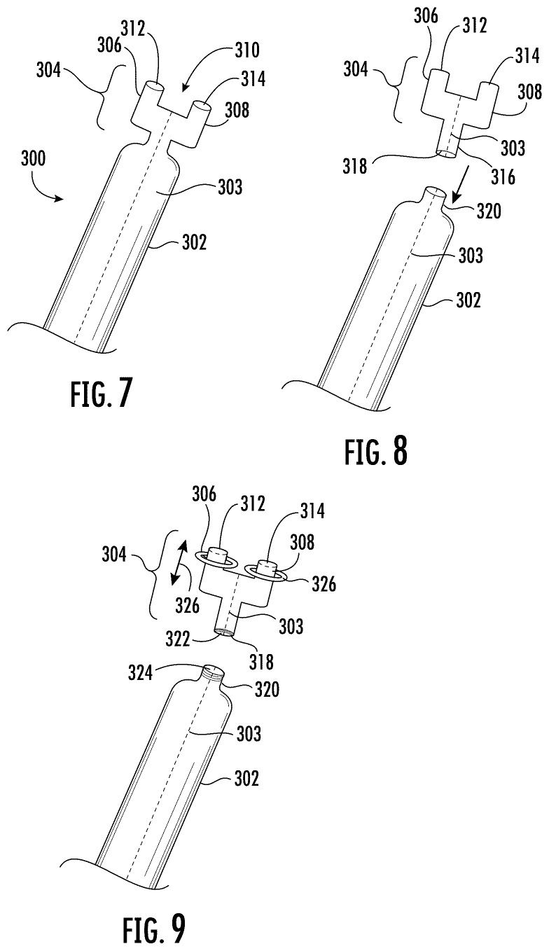

Referring to FIG. 7, the device for delivering a medication for the treatment or alleviation of one or more symptoms of a medical condition, referred to generally as nasal delivery device 300, is shown. The nasal delivery device 300 includes a body 302), configured to receive plunger 22. A fluid administering unit 304 (illustrated as being integrally formed as part of the body 302) is positioned at the opposite end of the nasal delivery device 300 from where the plungers 22 are inserted. The fluid administering unit 304 includes a first fluid delivery arm 306 and a second fluid delivery arm 308. The first fluid delivery arm 306 and the second fluid delivery arm 308 are separated by a gap 310. Each includes an opening 312, 314 at the end of the respective arm. The first fluid delivery arm 306 and a second fluid delivery arm 308 are configured to be inserted into or rest just outside of the nostril of the user, directing the fluid inside of the nostril through the openings 312 and 314. The nasal delivery device 300 is configured as a dual/double barrel unit (the dashed lines 303 illustrates an internal wall or divider to create the dual/double barrel body 302, extending to the fluid administering unit 304).

FIG. 8 provides an embodiment of the nasal delivery device 300 designed to allow for the fluid administering unit 304 to be attached and secured to the body 302. In this embodiment, the fluid administering unit 304 includes a body engaging member 316, illustrated as an elongated, having an internal section terminating in opening 318 sized and shaped to allow to receive and store therein a fluid administering unit receiving member 320, illustrated herein as an elongated body extending from the nasal delivery device body 302. The intersection between the body engaging member 316 and the fluid administering unit receiving member 320 may be that of a frictional fit but other securing/fastening mechanisms, such as chemical fastening may be used. Although shown as a single barrel, the nasal delivery device could be a double barrel, see the embodiment of the nasal delivery device 300 shown in FIG. 8 is also configured as a dual/double barrel unit (the dashed lines 303 illustrates an internal wall or divider to create the dual/double barrel body 302 and fluid administering unit 304).

FIG. 9 provides an embodiment of the nasal delivery device 300 designed to allow for the securing of the body engaging member 316 and the fluid administering unit receiving member 320 to be accomplished by via a mechanical mechanism, such as through threading. The internal section terminating in opening 318 may include threading 322 configured to correspond with the external threading 324 of the fluid administering unit receiving member 320. The first fluid delivery arm 306 and the second fluid delivery arm 308 may include a stopper disc 326, used to prevent or minimize the volume of back-flowing fluid (coming out of the user's nostrils). The topper disc 326 may be fixed in place, or configured to be movable (linearly, up/down), see double arrow 326. The embodiment of the nasal delivery device 300 shown in FIG. 9 is also configured as a dual/double barrel unit (the dashed lines 303 illustrates an internal wall or divider to create the dual/double barrel body 302 and fluid administering unit 304).

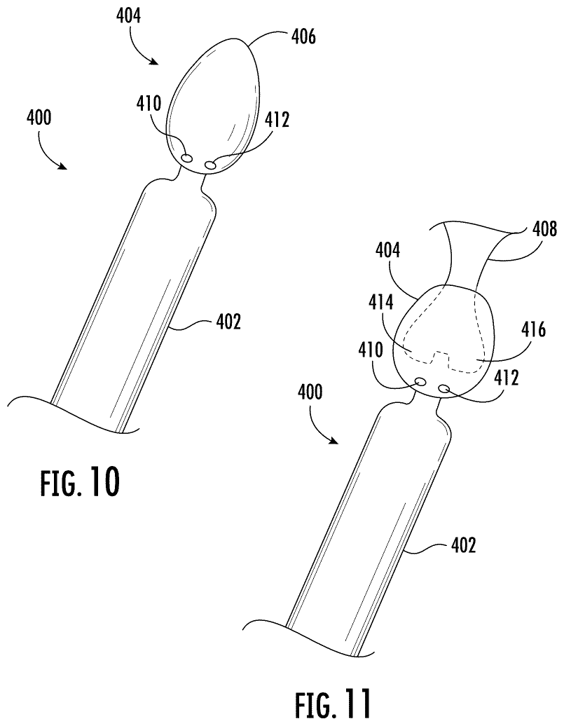

Referring to FIG. 10, the device for delivering a medication for the treatment or alleviation of one or more symptoms of a medical condition, referred to generally as nasal delivery device 400, is shown. The nasal delivery device 400 includes a body 402 (illustrated as a single barrel, but could be a double barrel), configured to receive plunger 22. A fluid administering unit 404 (illustrated as being integrally formed as part of the body 302 but could be independently attachable/removable) is positioned at the opposite end of the nasal delivery device 300 from where the plungers 22 are inserted. The fluid administering unit 404 includes a mask 406 (similar to CPR rescue masks (resuscitation mask). The mask 406 is sized and shaped to provide a tight fit on, and cover the nose 408, see FIG. 11. The mask 406 may include internal openings 410, 412, to direct the lidocaine into the nostrils 414, 416. Openings 410 and 412 may be spaced apart so that lidocaine can be dispensed into each individual nostril 414, 416. The nasal delivery device 400 is configured as a dual/double barrel unit.

In certain embodiments, the nasal delivery device 10, 100, 200, 300, or 400 may be part of a kit. The kit including one or more nasal delivery devices 10, 100, 200, 300, or 400, preloaded with lidocaine. The one or more nasal delivery devices 10, 100, 200, 300, or 400, where applicable, may include the fluid lines 86 attached thereto, or may include separate, individual fluid lines 86 which can be attached to the delivery derives by the user when needed.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures, and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A device for delivering a liquid agent into a user's nose for treating or alleviating one or more symptoms of a medical condition, comprising:
a double barrel delivery device body comprising a first fluid delivery member and a second fluid delivery member;
a first nasal adaptor plug fluidly connected to said first fluid delivery member, said first nasal adaptor plug comprising a body sized and shaped to engage with and occlude a user's first nostril, said body comprising an internal fluid passageway terminating in a fluid dispensing unit; and
a second nasal adaptor plug fluidly connected to said second fluid delivery member, said second nasal adaptor plug comprising a body sized and shaped to engage with and occlude a user's second nostril, said body comprising an internal fluid passageway terminating in a second fluid dispensing unit;
at least one moveable component that creates a force to expel said liquid agent from said double barrel delivery device body;
wherein, said liquid agent is delivered simultaneously to both nostrils or to one specific nostril, with no leakage or back flow.

2. The device according to claim 1, wherein at said least one moveable component that creates a force to expel said liquid agent from said double barrel delivery device body comprises:
a first plunger sized and shaped to fit and move within said first fluid delivery member; and
a second plunger sized and shaped to fit and move within said second fluid delivery member.

3. The device according to claim 1, wherein
said first fluid delivery member comprises an elongated body having a first end and a second end;
said second fluid delivery member comprises an elongated body having a first end and a second end;

4. The device according to claim 1, wherein said liquid agent is lidocaine.

5. The device according to claim 1, wherein the said liquid agent is pre-loaded into said first fluid delivery member and said second fluid delivery member.

6. The device according to claim 1, wherein said medical condition is a migraine.

7. The device according to claim 1, wherein said liquid agent is delivered to said user's sphenopalatine ganglion (SPG) deep to the floor of the nares/nostrils.

8. The device according to claim 1, wherein said first nasal adaptor plug and said second nasal adaptor plug each comprise a nipple or nozzle constructed and arranged for directed delivery of said agent.

9. The device according to claim 8, wherein said nipple or nozzle is made of a pliable material.

10. The device according to claim 1, wherein said first nasal adaptor plug and said second nasal adaptor plug are a made of a soft material conforming to a shape of said user's nostrils.

11. The device according to claim 1, wherein said first nasal adaptor plug and said second nasal adaptor plug are sized and shaped to prevent backflow or leakage of said agent during dispensing.

12. The device according to claim 1, wherein said first nasal adaptor plug and said second nasal adaptor plug are made of a flexible or pliable material.

13. A method of treating or alleviating of one or more symptoms of a migraine, comprising:
providing a delivery device comprising a first fluid delivery member and a second fluid delivery member; a first nasal adaptor plug fluidly connected to said first fluid delivery member, said first nasal adaptor plug comprising a body sized and shaped to engage with and occlude a user's first nostril, said body comprising an internal fluid passageway terminating in a fluid dispensing unit; a second nasal adaptor plug fluidly connected to said second fluid delivery member, said second nasal adaptor plug comprising a body sized and shaped to engage with and occlude a user's second nostril, said body comprising an internal fluid passageway terminating in a second fluid dispensing unit; and at least one moveable component that creates suction to draw in or expel said lidocaine from said delivery device, wherein, said lidocaine is delivered simultaneously to both nostrils or to one specific nostril, with no leakage or back flow.

14. The method according to claim 13, wherein said delivery device is pre-loaded with said lidocaine.

15. A kit for treating or alleviating of one or more symptoms of a migraine, comprising,
providing one or more delivery devices comprising a first fluid delivery member and a second fluid delivery member; a first nasal adaptor plug fluidly connected to said first fluid delivery member, said first nasal adaptor plug comprising a body sized and shaped to engage with and occlude a user's first nostril, said body comprising an internal fluid passageway terminating in a fluid dispensing unit; a second nasal adaptor plug fluidly connected to said second fluid delivery member, said second nasal adaptor plug comprising a body sized and shaped to engage with and occlude a user's second nostril, said body comprising an internal fluid passageway terminating in a second fluid dispensing unit; and at least one moveable component that creates suction to draw in or expel lidocaine from said delivery device, wherein, said lidocaine is delivered simultaneously to both nostrils or to one specific nostril, with no leakage or back flow; and
an effective amount of said lidocaine preloaded within said one or more delivery devices.
